(19) United States
(12) Patent Application Publication
(10) Pub. No.: US 2022/0170947 A1
(43) Pub. Date: Jun. 2, 2022

(54) COMPOSITIONS AND METHODS FOR DIAGNOSING URINARY TRACT OBSTRUCTION

(71) Applicant: The Research Institute at Nationwide Children's Hospital, Columbus, OH (US)

(72) Inventors: Christina B. Ching, Columbus, OH (US); Michael B. Becknell, Westerville, OH (US); Ashley R. Jackson, Grove City, OH (US); Sudipti Gupta, Columbus, OH (US); Janae Preece, Grosse Pointe Park, MI (US)

(21) Appl. No.: 17/625,267

(22) PCT Filed: Jul. 31, 2020
(86) PCT No.: PCT/US2020/044517

§ 371 Date: Jan. 6, 2022

Related U.S. Application Data
(60) Provisional application No. 62/881,450, filed on Aug. 1, 2019.

(51) Int. Cl. G01N 33/68

(52) U.S. Cl. CPC G01N 33/6893; G01N 2800/34

(57) ABSTRACT

A method of diagnosing and treating urinary tract obstruction that includes obtaining a urine sample from a human patient; detecting whether a predetermined uroepithelial peptide or predetermined urinary protein is present in the urine sample by either contacting the urine sample with an anti-uroepithelial peptide antibody and detecting binding between the uroepithelial peptide and the antibody or contacting the urine sample with an anti-urinary protein antibody and detecting binding between the urinary protein and the antibody; diagnosing the patient with urinary tract obstruction when the presence of either the predetermined uroepithelial peptide or predetermined urinary protein is detected; and operatively or non-operatively treating the patient to correct or remove the urinary tract obstruction.

### CROSS-REFERENCE TO RELATED APPLICATIONS

This application is a national stage application filed under 35 U.S.C. § 371 of International Patent Application No. PCT/US2020/0044517, filed on Jul. 31, 2020, which is incorporated by reference herein in its entirety for all purposes.

### STATEMENT REGARDING FEDERALLY FUNDED RESEARCH

This invention was made with government support under Grant No. DK125469 awarded by the National Institutes of Health. The U.S. government has certain rights in the invention.

### BACKGROUND

The described invention relates in general to proteins and other molecules that are or may be indicative of a particular medical condition or disease state, and more specifically to compositions and methods for diagnosing urinary tract obstruction based on the presence of certain peptides or proteins in urine samples.

Congenital obstructive uropathy is one of the most common causes of chronic kidney disease and need for renal transplantation in the pediatric population [1]. Timely diagnosis of urinary obstruction enables appropriate interventions with hopeful preservation of renal function and/or reversal of renal disease. Current diagnostics of urinary obstruction nearly universally rely on imaging results such as hydronephrosis on renal ultrasound and signs of delayed urinary drainage with renal dysfunction on nuclear scintigraphy. Yet, hydronephrosis is a nonspecific finding, detected in 1-5% of prenatal ultrasounds [2,3], representing a transient finding in the majority of cases that does not require intervention [4]. Nuclear scintigraphy itself can be difficult to interpret as it is dependent on adequate renal function, can be altered by capacious renal systems, and is prone to operator variability [5]. While dynamic magnetic resonance technology is emerging as a diagnostic tool, it is limited in its accessibility.

In an effort to improve current diagnostic modalities, urinary biomarkers have been evaluated for their potential use in identifying clinically significant urinary tract obstruction. Serum markers such as creatinine are currently utilized; however, this marker is often normal in patients with a healthy contralateral kidney, limiting its application in patients with unilateral obstruction [6,7]. Urinary biomarkers such as neutrophil gelatinase-associated lipocalin (NGAL) and human hepatocarcinoma-intestine-pancreas/pancreatitis-associated protein (HIP/PAP) have been identified as indicators of injury occurring in the urinary tract. However, only NGAL has been recognized in the literature as a urinary biomarker of urinary tract obstruction and its ability to normalize after surgical removal of the obstruction has not been characterized in a consistent manner. Certain other proteins have been detected during urinary tract infection or inflammation but have not been detected during anatomic or functional obstruction of the urinary tract. Accordingly, there is an ongoing need for a biomarker-based, non-invasive method for detecting and monitoring patients for urinary tract obstruction, wherein the method could be used alone or to augment current imaging techniques that are limited by their own sensitivity, specificity and invasiveness (e.g., by requiring IVs, catheters, and exposure to radiation).

### SUMMARY

The following provides a summary of certain exemplary embodiments of the present invention. This summary is not an extensive overview and is not intended to identify key or critical aspects or elements of the present invention or to delineate its scope. However, it is to be understood that the use of indefinite articles in the language used to describe and claim the present invention is not intended in any way to limit the described system. Rather the use of “a” or “an” should be interpreted to mean “at least one” or “one or more”.

Disclosed are compositions and methods for quantitative detection of urinary proteins for providing a noninvasive method for detecting injury to the lining of the urinary tract (uroepithelium) as a consequence of functional or anatomic urinary tract obstruction. Measurement of urinary proteins in individuals at risk for developing urinary tract obstruction facilitates identification of subclinical injury to the urinary tract. Additionally, quantification of such urinary proteins facilitates decision making regarding operative versus conservative, non-operative management of patients with radiographic evidence of urinary tract obstruction. Finally, quantification of these urinary proteins functions as a marker for following postoperatively and monitoring surgical success.

In one implementation, a method of detecting at least one uroepithelial peptide in a patient is provided, wherein the presence of the uroepithelial peptide is indicative of urinary tract obstruction. This method comprises obtaining a urine sample from a human patient; and detecting whether a predetermined uroepithelial peptide is present in the urine sample by contacting the urine sample with an anti-uroepithelial peptide antibody and detecting binding between the uroepithelial peptide and the antibody. The uroepithelial peptide may be β defensin 1 (BD-1); human α defensin 5 (HD-5); hepatocarcinoma-intestine-pancreas/pancreatitis-associated protein (HIP/PAP); cathelicidin LL-37; neutrophil gelatinase-associated lipocalin (NGAL); or combinations thereof. The method may further comprise measuring the amount of uroepithelial peptide in the urine sample using quantitative enzyme-linked immunosorbent assay (ELISA).

In another implementation, a method of detecting a urinary protein in a patient is provided, wherein the presence of the urinary protein is indicative of urinary tract obstruction. This method comprises obtaining a urine sample from a human patient; and detecting whether a predetermined urinary protein is present in the urine sample by contacting the urine sample with an anti-urinary protein antibody and detecting binding between the urinary protein and the antibody. The urinary protein may be UPK3A, UPK2, UPK20, UPK14, or combinations thereof. The method may further comprise measuring the amount of urinary protein in the urine sample using quantitative enzyme-linked immunosorbent assay (ELISA).

In yet another implementation, a method of diagnosing and treating urinary tract obstruction is provided. This method comprises obtaining a urine sample from a human patient; detecting whether a predetermined uroepithelial peptide or predetermined urinary protein is present in the urine sample by either contacting the urine sample with an anti-uroepithelial peptide antibody and detecting binding between the uroepithelial peptide and the antibody or contacting the urine sample with an anti-urinary protein antibody and detecting binding between the urinary protein and the antibody; diagnosing the patient with urinary tract obstruction when the presence of either the predetermined uroepithelial peptide or predetermined urinary protein is detected; and operatively or non-operatively treating the patient to correct or remove the urinary tract obstruction. The uroepithelial peptide may be β defensin 1 (BD-1); human α defensin 5 (HD-5); hepatocarcinoma-intestine-pancreas/pancreatitis-associated protein (HIP/PAP); cathelicidin LL-37; neutrophil gelatinase-associated lipocalin (NGAL); or combinations thereof. The urinary protein may be UPK3A, UPK2, UPK20, UPK 14, or combinations thereof. The method may further comprise measuring the amount of uroepithelial peptide or the amount of urinary protein in the urine sample using quantitative ELISA. The method further comprises confirming the effective removal of the urinary tract obstruction by obtaining a second, post-treatment urine sample from the patient and testing the second urine sample to confirm an absence of or significant reduction in the amount of urinary protein.

Additional features and aspects of the present invention will become apparent to those of ordinary skill in the art upon reading and understanding the following detailed description of the exemplary embodiments. As will be appreciated by the skilled artisan, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description given below, serve to explain the principles of the invention, and wherein:

FIGS. 1A-D depict the results of a series of ELISAs illustrating urinary AMP expression in patients before (Pre) surgical correction of ureteropelvic junction obstruction (UPJO) and after (Post) surgical correction of UPJO, wherein FIG. 1A depicts the results of a HIP/PAP ELISA; FIG. 1B depicts the results of a BD-1 ELISA; FIG. 1C depicts the results of a NGAL-37 ELISA; and FIG. 1D depicts the results of an LL-37 ELISA (with * and ** equaling statistical significance wherein *p=0.0215 and **p=0.0052);

FIGS. 2A-D depict the results of a series of ELISAs illustrating comparatively the urinary AMP levels in individual patients before (Pre) surgical correction of ureteropelvic junction obstruction (UPJO) and after (Post) surgical correction of UPJO, wherein FIG. 1A depicts the results of a HIP/PAP ELISA; FIG. 1B depicts the results of a BD-1 ELISA; FIG. 1C depicts the results of a NGAL-37 ELISA; and FIG. 1D depicts the results of an LL-37 ELISA;

FIGS. 3A-3B depict the results of a series of ELISAs illustratively comparing urinary BD-1 and HIP/PAP between patients who have been successfully unobstructed (Post) and age- and sex-matched healthy controls (Control), wherein FIG. 1A depicts the results of a BD-1 ELISA; FIG. 1B depicts the results of a HIP/PAP ELISA;

FIG. 4A depicts the results of experiments demonstrating elevated levels of urinary UPK3A and UPK2 in patients with ureteropelvic junction obstruction (UPJO) versus unobstructed controls (with * representing statistical significance);

FIG. 4B and FIG. 4C depict the results of experiments demonstrating elevated levels of urinary UPK20 (FIG. 4B) and UPK14 (FIG. 4C) respectively in patients with ureteropelvic junction obstruction (UPJO) versus unobstructed controls; and

FIG. 5 depicts the results of experiments demonstrating elevated levels of urinary HIP/PAP in patients with neurogenic bladder (NGB) compared to healthy controls (with * representing statistical significance).

### DETAILED DESCRIPTION

Exemplary embodiments of the present invention are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the invention. Accordingly, the following embodiments of the invention are set forth without any loss of generality to, and without imposing limitations upon, the claimed invention.

Biomarkers are typically defined as objective, quantifiable characteristics of biological processes and may be one or more biological molecules found in blood, other body fluids, or tissues that are a sign of a normal or abnormal (e.g. pathogenic) process, or of a condition or disease. Biomarkers may be used to determine the effectiveness of a treatment (e.g., pharmacologic response to a therapeutic intervention) for a particular disease or medical condition. A biomarker may also refer to a specific protein or group of proteins, the presence and/or concentration of which may indicate the presence or severity of a disease state. Biomarkers may be detectable and measurable by a variety of methods including physical examination, laboratory assays, and medical imaging.

Antimicrobial peptides (AMPs) exert bactericidal and bacteriostatic activity that promotes urinary tract sterility. Other scientific evidence suggests that AMPs may also be present in noninfectious conditions, thereby potentially reflecting broader functionality during uroepithelial adaptation and regeneration following injury. The compositions and methods disclosed herein provide a panel of urinary tract epithelial derived biomarkers (e.g., uroepithelial AMPs and urinary structural proteins) in the context of anatomic or functional urinary tract obstruction. These compositions and methods were useful for identifying significant differences in urinary protein biomarkers in pediatric patients undergoing operative repair of ureteropelvic junction obstruction as compared to healthy, unobstructed control patients. A significant decrease in a subset of these urinary protein biomarkers in pediatric patients with successful correction of their ureteropelvic junction obstruction was observed using the disclosed methods, as were significant differences in urinary protein biomarkers in pediatric patients with neurogenic bladder due to spinal dysraphism as compared to healthy control patients.

### AMPs as Biomarkers

The following experiments and data are illustrative with regard to the disclosed invention, as are the methods and results disclosed in Gupta S, et al. Urinary antimicrobial peptides: potential novel biomarkers of obstructive uropathy. J Pediatr Urol 14 (3): 238.el-238.e6. PMID 29706289; and Gupta S et al., Impact of successful pediatric ureteropelvic junction obstruction surgery on urinary HIP/PAP and BD-1 levels, J Pediatr Urol, https://doi.org/10.1016/j.jpurol.2020.03.006, which are incorporated by reference herein, in their entirety for all purposes. Data disclosed herein was derived from ELISA-based detection of urinary proteins using commercially available individual ELISA kits. In other implementations, individual tests are consolidated into a single panel of urinary analytes using a multiplex bead-based immunoassay for rapid quantification of multiple urinary protein analytes, thereby reducing cost, time, and urine sample volume. Because urine pH and osmolality vary widely in various physiologic and pathologic states, in some implementations, detection conditions have been optimized to accommodate urine pH ranging from 5-9 and osmolalities ranging from 50-1200 mOsm/kg.

In the pediatric patient whose ureteropelvic junction obstruction (UPJO) is not always symptomatic, imaging is the most common means of detecting surgical success. There is interest, however, in other means of post-operative monitoring. Certain AMPs were previously found to be elevated in UPJO, but the impact of surgical correction on these AMPs was unknown. An objective of the research described herein was to determine if elevated levels of candidate urinary AMP biomarkers of urinary tract obstruction decrease following UPJO repair.

AMPs are small, cationic peptides normally produced as part of the innate immune system by the urothelium, renal intercalated cells, and phagocytes in response to urinary tract infection [8,9]. AMPs are expressed in non-infectious states such as hemorrhagic cystitis and acute kidney injury suggesting they may be broader markers of urinary tract pathology [10,11]. Certain AMPs have been found to be elevated in uninfected patients with unilateral UPJO requiring surgery as compared to age- and sex-matched healthy control patients [12], suggesting urinary AMP levels may reflect changes in the uroepithelial barrier that could be altered in the setting of obstruction and thus could be used as markers of significant obstruction. The research discussed herein investigated the expression of these AMPs after successful correction of obstruction in a subset of patients who are at least six months out from surgical repair. This research evaluated the post-operative expression of these same AMPs after successful surgical correction. This research also compared the postoperative expression of any decreased AMPs to that of control patients without a history of obstruction to determine if these values return to normal with the expectation that elevated levels of markers in obstruction would decrease and return to normal when the obstruction was relieved.

### Materials and Methods

Pediatric patients undergoing surgical correction of UPJO were recruited for study participation. Bladder urine from uninfected consenting/assenting patients was collected immediately prior to surgery and then at least six months afterward. Based on prior studies demonstrating significant elevation of beta defensin 1 (BD-1), hepatocarcinoma-intestine-pancreas/pancreatitis-associated protein (HIP/PAP), cathelicidin (LL-37), and neutrophil gelatinase associated lipocalin (NGAL) in patients with UPJO versus control patients, enzyme linked immunosorbent assays (ELISAs) were performed on these four AMPs to compare expression thereof before and after surgical intervention. If found to significantly decrease, AMP levels were compared to healthy controls. AMP levels were normalized to urine creatinine. Results were analyzed with paired t test or Wilcoxon test using Graphpad software. Correlation was calculated using Pearson or Spearman correlation. A p-value of <0.05 was considered significant.

Thirteen UPJO patients were included in this study; 9 were male (69%). Age at surgery was a median of 4.3 years (average 6.1, range 0.4-18.4 years). Follow-up urine samples were collected a median of 27.4 months after surgery (average 27.4; range 7.8-45.3 months). All thirteen patients exhibited clinical improvement and/or signs of improved hydronephrosis on postoperative imaging. HIP/PAP and BD-1 significantly decreased in post-surgical samples compared to presurgical samples (p=0.02 and 0.01, respectively); NGAL and LL-37 did not significantly change. Overall, HIP/PAP decreased in 12 patients (92%) and BD-1 decreased in 11 patients (85%). BD-1 levels after successful repair were not different from healthy controls (p=0.06).

Urinary biomarkers of obstruction are expected to detect significant obstructive pathology as well as reflect its resolution, thereby enabling their use in postoperative monitoring and augmenting current methods of determining successful surgical outcome through imaging. The AMPs HIP/PAP and BD-1 are significantly elevated in UPJO but then significantly decrease after pyeloplasty, with BD-1 returning to healthy control levels. Accordingly, these AMPs are useful as markers of successful surgical intervention. Just as elevated urinary HIP/PAP and BD-1 levels suggest clinically significant upper urinary tract obstruction, their decrease suggests successful surgical intervention. Potentially, this could have implications for simplifying the required post-operative monitoring and follow-up of patients after pyeloplasty.

Previously collected aliquots of bladder urine samples from consenting/assenting pediatric patients (18 years old) at time of pyeloplasty for presumed UPJO were used [12]. These samples represent a “Pre” group. Follow-up bladder urine samples were collected in the same patients at least 6 months after surgery and all drainage tubes (e.g. stents, nephrostomy tubes, catheters) were removed. Time of collection was based on when any tubes were removed to eliminate the possibility that inflammation or urothelial changes directly in response to a foreign body could artificially alter AMP expression. These samples represent a “Post” group. These samples were collected by either clean catch or bagged urine. Patients were only included if follow-up urine samples were obtained >6 months from surgery and removal of all urinary drains. Patients were excluded from analysis if they demonstrated signs of UTI on urinalysis (such as anything more than trace leukocytes or positive nitrites) and/or symptomatology at time of collection. The electronic medical record was reviewed to identify any concern for persistent symptoms after surgery. All imaging was reviewed including most recent post-operative renal ultrasound and nuclear scintigraphy, if present. A single reviewer (C.B.C.) read and graded renal ultrasounds for hydronephrosis by the Society for Fetal Urology (SFU) system. Patients were included regardless of concern for re-obstruction based on imaging or symptoms.

A selection of control urine aliquots from healthy pediatric patients were used for comparison to the “Post” group. The “Control” samples chosen were age- and sex-matched to the patients from whom “Post” samples had been obtained. Patients were matched one-to-one as close to the exact age of the Post patient, but up to a 1-year difference in age was allowed. These samples were bladder urine collected by means of free void or bagged sample. Urinalysis of samples was performed by dipstick (Siemens Healthcare Diagnostics, Inc., Tarrytown N.Y.) immediately after collection, after which a protease inhibitor (Assay Assure, Sierra Molecular, Incline Village Nev.) was added. Samples were then processed, aliquoted, and stored at 80° C. until analysis as previously described [12]. Samples were restricted to a single freeze/thaw cycle before analysis.

Based on previous results indicating that the AMPs beta defensin 1 (BD-1), hepatocarcinomatous-intestine-pancreas/pancreatitis-associated protein (HIP/PAP), cathelicidin (LL-37), and neutrophil gelatinase-associated lipocalin (NGAL) were significantly elevated in patients with UPJO compared to controls [12], enzyme-linked immunosorbent assays (ELISA) were performed on these four AMPs to compare their expression before (Pre) and after (Post) surgical intervention. BD-1 was run at 1:1000 dilution (Peprotech, Rocky Hill, N.J.) while HIP/PAP (Fisher Scientific, Pittsburgh, Pa.), LL-37 (Hycult Biotech, Plymouth Meeting, Pa.), and NGAL (Hycult Biotech, Plymouth Meeting, Pa.) were all run undiluted. ELISAs were performed in duplicate on cell-free supernatants. Due to observed variability between ELISA plate readings, corresponding comparison groups were run on the same ELISA plate. AMP levels were normalized to urine creatinine (UCr) as previously described [12] and expressed as a ratio to UCr (ng/mg). D'Agostino-Pearson omnibus normality test determined if samples were parametric. Paired sample analysis was performed by paired t test or Wilcoxon test depending if samples were parametric or nonparametric, respectively. Pearson or Spearman correlation was used to evaluate relationships between variables. Indicated statistical analyses were performed using GraphPad (La Jolla, Calif.). In all cases, p<0.05 was considered statistically significant.

Of the 30 patients included in earlier research, follow-up urine samples >6 months after surgery for 13 of these patients were collected. Reasons for inability to collect follow-up samples on the other 17 patients included lack of follow-up, follow-up at an off-site location that precluded being able to properly collect and process urine for analysis, and inability of the patient or unwillingness to provide a urine sample at follow-up visit. Patients had undergone prior pyeloplasty for UPJO ultimately at the discretion of the surgical provider but for such indications as worsening hydronephrosis on renal ultrasound, decline in ipsilateral relative renal function on renal scintigraphy, and for symptoms such as pain. One of the patients had a functionally solitary kidney while all others had a normal contralateral kidney. All thirteen patients had clinical improvement from before surgery and/or signs of improved hydronephrosis on post-operative imaging. One patient with symptom improvement did not have imaging available for review. None of the patients had preoperative urinary tract infections; only one patient had a post-operative urinary tract infection, 11 months before their Post sample was collected.

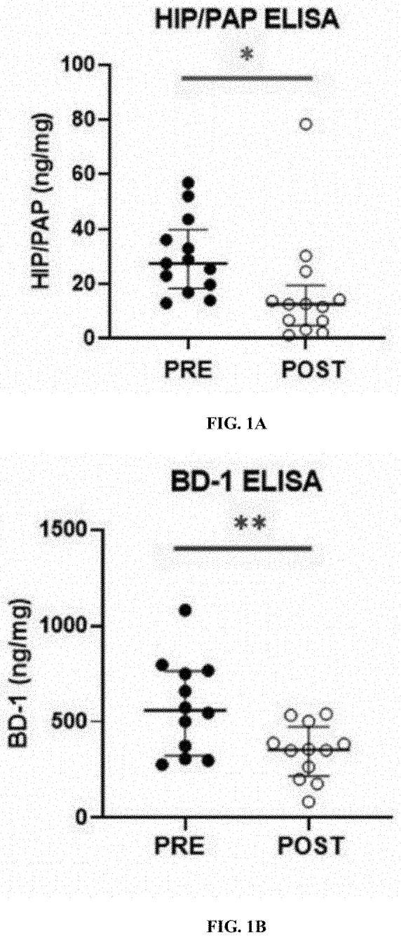

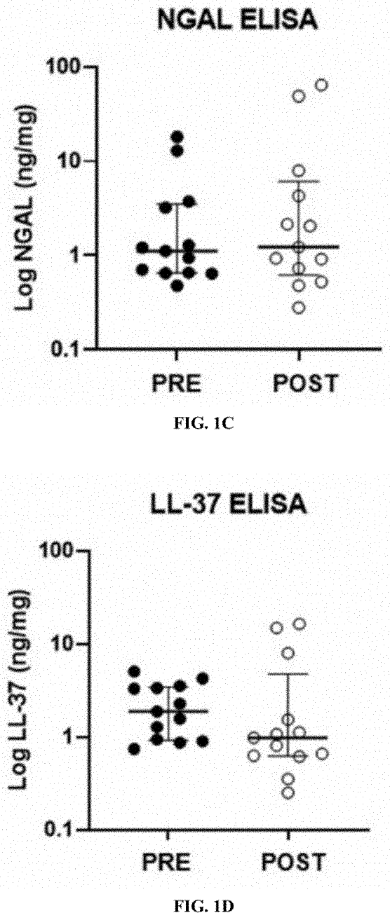

As shown in FIGS. 1A-1D, HIP/PAP and BD-1 significantly decreased in post-surgical samples (Post) compared to pre-surgical samples (Pre) (p=0.0215 and 0.0052, respectively). NGAL and LL-37 did not significantly change (p >0.9999 and p=0.5417, respectively). A correlation between NGAL and Post SFU grade was investigated based on a previously identified correlation between Pre SFU grade and NGAL [12]. There was no significant correlation (p=0.653, Spearman correlation). Interestingly, in the patient with a functionally solitary kidney, all levels of markers decreased post-operatively.

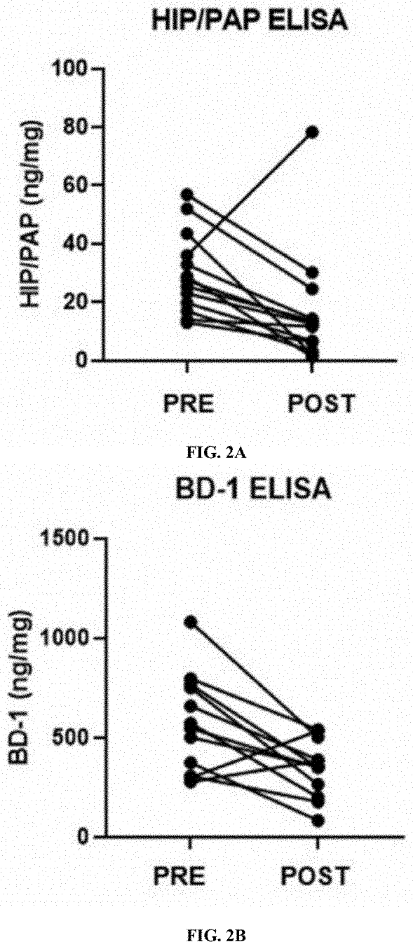

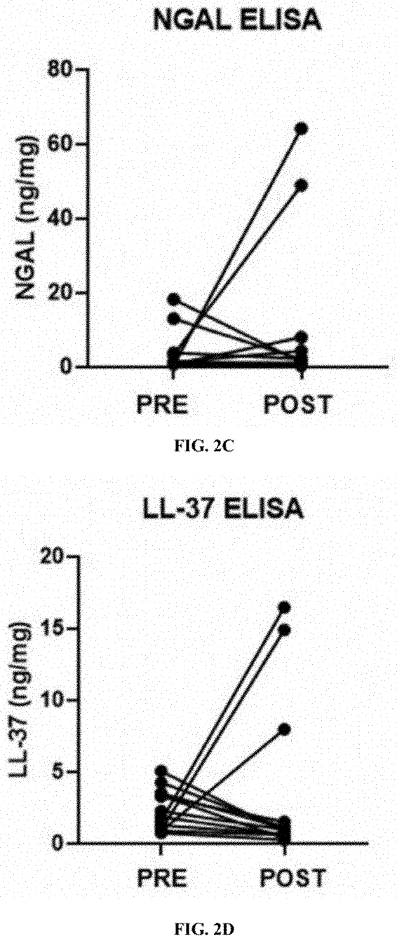

In evaluating values of each individual Pre and Post surgery, there were only two individuals with a rise in HIP/PAP and/or BD-1 after surgery (see FIGS. 2A-2D). There was no obvious unifying factor to explain the observed rise in values: one was a 7-year-old male with an 18% functioning obstructed kidney while the other was an 18-year-old female with a 43% functioning obstructed kidney. Neither patient had a history of UTIs or a post-surgical renal scan to evaluate function. Both had improved SFU grade after surgery on post-operative renal ultrasound. Using the findings that BD-1 increased in 2 patients and HIP/PAP increased in only 1 after surgery, the specificity of BD-1 was calculated at 85% and that of HIP/PAP was calculated at 92%. Given none of the patients had evidence of failed pyeloplasty (true positives for disease), it was not possible to calculate a sensitivity for the markers or perform receiver operating characteristic curves.

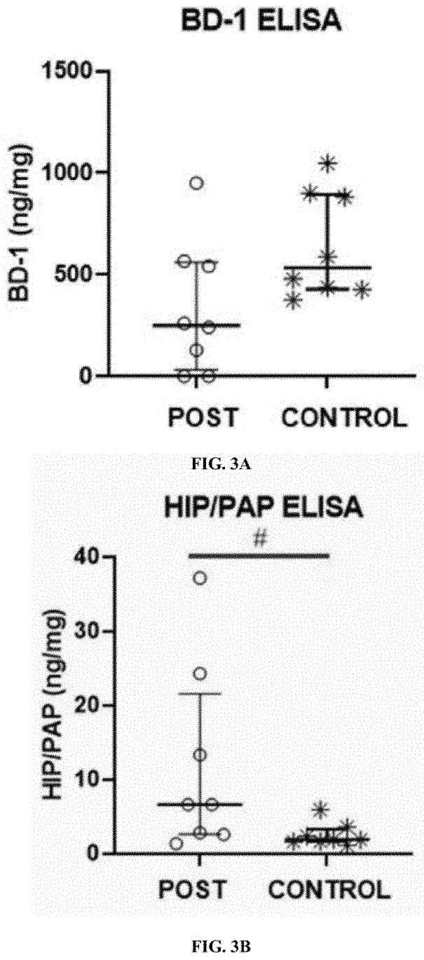

Since HIP/PAP and BD-1 were the only two AMPs to significantly decrease in the Post samples, only these two AMPs were compared to Control urine samples. Because it had previously been found that HIP/PAP expression correlated with age [12], eight of the Post urine were age- (p=0.9591) and sex- (p=0.5896) matched samples to eight of the previously collected Control urine samples. BD-1 Post urine samples normalized to the cohort of healthy control urine samples while HIP/PAP did not (see FIG. 3A-3B). There were only 2 individuals with post-operative nuclear scintigraphy demonstrating renal function, thus limiting the ability to check a correlation between postoperative biomarker values and their corresponding renal function. Given the variability in time from surgery, a correlation in expression of the relevant AMPs with time was assessed. A significant correlation was not found between Post urine levels of HIP/PAP, BD-1, NGAL, or LL-37 and time from surgery (p=0.591 (Spearman's correlation); p=0.449 (Pearson's correlation); p=0.176 (Spearman's correlation); and p=1.000 (Spearman's correlation), respectively).

Current tools for evaluation of successful surgical intervention of UPJO include imaging and clinical presentation. Pediatric patients are not always symptomatic from their UPJO to enable determination if resolution signifies improvement of obstruction and there are limitations associated with post-operative imaging [13,14]. As such, an alternative and objective means of tracking an obstructive process in the urinary tract is attractive.

There has been great interest in identifying urinary biomarkers of pediatric urinary tract obstruction to supplement if not replace current diagnostic methods as minimally invasive alternatives to current radiologic tests [15]. The AMPs HIP/PAP, BD-1, NGAL, and LL-37 were previously found to be significantly elevated in children with UPJO compared to age- and sex-matched controls [12], suggesting that urinary AMPs may represent novel markers of urinary tract obstruction. Given a good biomarker should also reflect its resolution [16], a subsequent study was performed to determine whether successful surgical repair led to a decrease and even normalization of HIP/PAP, BD-1, NGAL, and LL-37 urinary levels.

Of the four initial biomarkers, two suggested clinical viability. HIP/PAP and BD-1 significantly decreased after successful surgical correction with relatively high specificity for surgical success. Unfortunately, due to the lack of surgical failures, it was not possible to evaluate the sensitivity of these markers, and thus their predictive value. HIP/PAP is made specifically by the urothelium [11,17] while BD-1 is primarily made by the renal distal tubules and collecting duct [18,19]. Given urinary tract obstruction is known to result in epithelial injury [20,21], it seems appropriate that peptides made by these cells may be released in the setting of obstructive uropathy. Just as their elevated expression could be a marker of obstruction, their declining expression post-operatively could be a marker of surgical success—suggesting HIP/PAP and BD-1 are candidate biomarkers for UPJO diagnosis and repair. The ability of these markers to decrease does speak towards the capability of urinary tract epithelial cells to remodel and even repair once obstruction is corrected, particularly in the case of BD-1 levels that in fact normalize to that of healthy controls.

Conversely, NGAL and LL-37, which had previously been identified as elevated in UPJO patients [12] did not significantly decrease post-operatively. NGAL serves as a marker of renal injury as a result of both increased distal nephron gene expression and altered proximal tubule reabsorption [22-24]. LL-37 is expressed by urinary tract epithelial cells [25]. The persistent elevation of these markers suggests there may be an element of irreversible or ongoing tubular damage despite apparent surgical success based on ultrasound imaging and clinical history. NGAL has been demonstrated to decrease significantly as early as one month after successful UPJO repair [26,27]. Its ability to normalize however is somewhat mixed with two studies finding that urine NGAL levels normalize to levels detected in controls by 6 months after surgery and remains persistently low 1 and 3 years after surgery [24,26]. Another, however, found that while NGAL significantly decreases 3 months after surgery (p<0.05), it is still higher than in healthy children (p>0.05) [28]. Xie et al. found that urinary NGAL levels measured 72 h after surgical correction of obstructing nephrolithiasis discriminated patients with deteriorated versus stable renal function one year later [29]. Their study is novel in that it found NGAL could predict those with irreversible renal dysfunction post-operatively; prior work had simply demonstrated NGAL levels inversely correlate with the relative function of the affected kidney at the time of collection [22,28,30]. Future studies evaluating patients at the time of pyeloplasty for UPJO may evaluate if urinary NGAL levels could predict functional recovery in patients, particularly if an indication for intervention was diminished renal function on nuclear scintigraphy.

### Proteins as Biomarkers

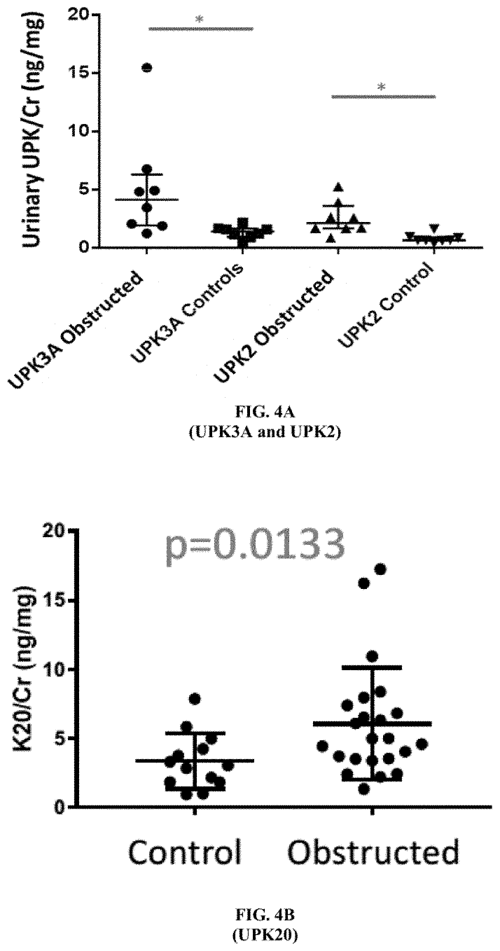

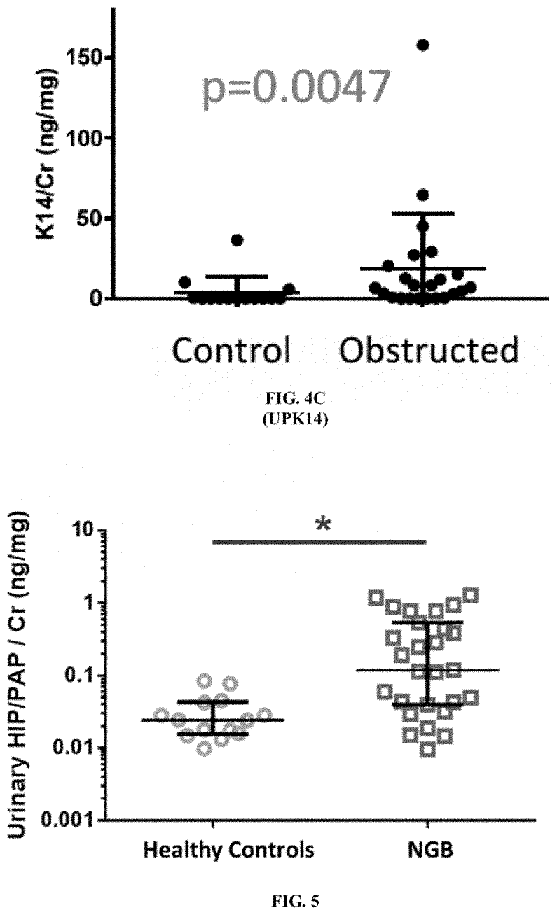

In addition to AMPS, proteins (e.g., keratins) involved with uroepithelial integrity were also shown to be increased in children having obstructive uropathy. Further experimentation analyzed the expression of urinary proteins UPK3A, UPK2, UPK20, and UPK14 in children undergoing surgical intervention for ureteropelvic junction obstruction (UPJO) compared to unobstructed controls. Significantly higher expression of UPK3A (p=0.005), UPK2 (p=0.0006), K20 (p=0.0133), and K14 (p=0.0047; Mann-Whitney U test) was observed in patients having UPJO at the time of surgical decompression. FIG. 4A depicts the results of experiments demonstrating elevated levels of urinary UPK3A and UPK2 in patients with ureteropelvic junction obstruction (UPJO) versus unobstructed controls (with * representing statistical significance); and FIG. 4B and FIG. 4C depict the results of experiments demonstrating elevated levels of urinary UPK20 and UPK14 respectively in patients with ureteropelvic junction obstruction (UPJO) versus unobstructed controls. These results suggest that urinary structural proteins, such as UPK3A, UPK2, UPK20, UPK14, individually or in combination with one another, may serve as biomarkers of uroepithelial injury.

Experiments were also performed to determine if the observation of elevated uroepithelial proteins was applicable to patients having functional urinary tract obstruction as a consequence of spinal cord injury. A study that included twenty-six patients having neurogenic bladder (NGB) as a consequence of spina bifida identified significantly elevated urinary HIP/PAP levels in children with NGB compared to healthy pediatric controls (p=0.0005; Mann-Whitney test). FIG. 5 depicts the results of experiments demonstrating elevated levels of urinary HIP/PAP in patients with neurogenic bladder (NGB) compared to healthy controls (with * representing statistical significance). These results suggest that uroepithelial proteins, such as HIP/PAP, may accumulate in urine of children with abnormal bladder dynamics.

The data disclosed herein supports the conclusion that elevations in urinary levels of uroepithelial proteins, such as AMPs and uroplakins, may serve as biomarkers of obstructive uropathy. The disclosed compositions provide an array of urinary biomarkers for detecting urinary tract obstruction and may be used in urine-based assays for diagnosing or risk-stratifying urinary tract obstruction. Results of these urine-based assays may be used to guide the management and follow-up of urinary tract obstruction and to specifically assist urologists and nephrologists in decision-making regarding which patients require more frequent or invasive serial imaging, additional laboratory monitoring, and, most critically, which patients require operative (i.e., surgical) management. This invention may also provide predictive value in identifying patients who require operative repair of urinary tract obstruction or at risk for urinary tract decompensation as a result of functional obstruction.

Other implementations utilize the disclosed compositions and associated methods for distinguishing patients who require operative management of urinary tract obstruction from those patients who can be managed non-operatively. Urine samples collected from patients with prenatal hydronephrosis may be used to determine if urinary protein biomarkers are predictive of which patients will exhibit clinically significant hydronephrosis compared to patients in whom the condition will spontaneously resolve. Additionally, urinary protein biomarkers can be used to distinguish various types of urinary tract obstruction (e.g., UPJO versus posterior urethral valves) and whether biomarker levels differ with respect to other structural abnormalities of the urinary tract, such as primary vesicoureteral reflux.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated references and similar materials differs from or contradicts this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about” used throughout this specification are used to describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed inventive subject matter. Various functions and elements described herein may be partitioned differently from those shown without departing from the scope of the disclosed inventive subject matter. Generic principles defined herein may be applied to other implementations. Different numbers of a given module or unit may be employed, a different type or types of a given module or unit may be employed, a given module or unit may be added, or a given module or unit may be omitted.

It should be appreciated that all combinations of the foregoing concepts and additional concepts discussed in greater detail herein (provided such concepts are not mutually inconsistent) are contemplated as being part of the disclosed inventive subject matter. In particular, all combinations of claimed subject matter appearing at the end of this disclosure are contemplated as being part of the inventive subject matter disclosed herein. While the disclosed inventive subject matter has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed inventive subject matter in its broader aspects is not limited to any of the specific details, representative devices and methods, and/or illustrative examples shown and described. Accordingly, departures may be made from such details without departing from the spirit or scope of the general inventive concept.

The following references form part of the specification of the present application and each reference is incorporated by reference herein, in its entirety, for all purposes.
[1] Smith J M, Stablein D M, Munoz R, Hebert D, McDonald R A. Contributions of the transplant registry: the 2006 annual report of the North American pediatric renal trials and collaborative studies (NAPRTCS). Pediatr Transplant 2007; 11(4):366e73.
[2] Ismaili K, Hall M, Donner C, Thomas D, Vermeylen D, Avni F E. Results of systematic screening for minor degrees of fetal renal pelvis dilatation in an unselected population. Am J Obstet Gynecol 2003; 188(1):242e6.
[3] Livera L N, Brookfield D S, Egginton J A, Hawnaur J M. Antenatal ultrasonography to detect fetal renal abnormalities: a prospective screening programme. BMJ 1989; 298(6685):1421e3.
[4] Nguyen H T, Herndon C D, Cooper C, Gatti J, Kirsch A, Kokorowski P, et al. The Society for Fetal Urology consensus statement on the evaluation and management of antenatal hydronephrosis. J Pediatr Urol 2010; 6(3):212e31.
[5] Perez-Brayfield M R, Kirsch A J, Jones R A, Grattan-Smith J D. A prospective study comparing ultrasound, nuclear scintigraphy and dynamic contrast enhanced magnetic resonance imaging in the evaluation of hydronephrosis. J Urol 2003; 170(4 Pt 1): 1330e4.
[6] Mandell J, Peters C A, Estroff J A, Allred E N, Benacerraf B R. Human fetal compensatory renal growth. J Urol 1993; 150(2 Pt 2):790e2.
[7] Peters C A, Gaertner R C, Carr M C, Mandell J. Fetal compensatory renal growth due to unilateral ureteral obstruction. J Urol 1993; 150(2 Pt 2):597e600.
[8] Ching C, Schwartz L, Spencer J D, Becknell B. Innate immunity and urinary tract infection. Pediatr Nephrol 2019. In press.
[9] Spencer J D, Schwaderer A L, Becknell B, Watson J, Hains D S. The innate immune response during urinary tract infection and pyelonephritis. Pediatr Nephrol 2014; 29(7): 1139e49.
[10] Lucarelli G, Mancini V, Galleggiante V, Rutigliano M, Vavallo A, Battaglia M, et al. Emerging urinary markers of renal injury in obstructive nephropathy. Biomed Res Int 2014; 2014:303298.
[11] Takahara Y, Suzuki A, Maeda M, Kawashima H, Nakatani T, Kiyama H. Expression of pancreatitis associated proteins in urothelium and urinary afferent neurons following cyclophosphamide induced cystitis. J Urol 2008; 179(4):1603e9.
[12] Gupta S, Jackson A R, DaJusta D G, McLeod D J, Alpert S A, Jayanthi V R, et al.

Urinary antimicrobial peptides: potential novel biomarkers of obstructive uropathy. J Pediatr Urol 2018; 14(3):238 e1e6.
[13] Castagnetti M, Novara G, Beniamin F, Vezzu B, Rigamonti W, Artibani W. Scintigraphic renal function after unilateral pyeloplasty in children: a systematic review. BJU Int 2008; 102(7): 862e8.
[14] Lam W, Fernando A, Issa R, Heenan S, Sandhu S, Le Roux P, et al. Is routine postoperative diuresis renography indicated in all adult patients after pyeloplasty for ureteropelvic junction obstruction? Urology 2015; 85(1):246e51.
[15] Papachristou F, Pavlaki A, Printza N. Urinary and serum biomarkers in ureteropelvic junction obstruction: a systematic review. Biomarkers 2014; 19(7):531e40.
[16] Vaidya V S, Ramirez V, Ichimura T, Bobadilla N A, Bonventre J V. Urinary kidney injury molecule-1: a sensitive quantitative biomarker for early detection of kidney tubular injury. Am J Physiol Ren Physiol 2006; 290(2):F517e29.
[17] Spencer J D, Jackson A R, Li B, Ching C B, Vonau M, Easterling R S, et al. Expression and significance of the HIP/PAP and RegIIIgamma antimicrobial peptides during mammalian urinary tract infection. PloS One 2015; 10(12): e0144024.
[18] Schnapp D, Reid C J, Harris A. Localization of expression of human beta defensin-1 in the pancreas and kidney. J Pathol 1998; 186(1):99e103.
[19] Valore E V, Park C H, Quayle A J, Wiles K R, McCray P B, Ganz T. Human beta-defensin-1: an antimicrobial peptide of urogenital tissues. J Clin Invest 1998; 101(8):1633e42.
[20] Jackson A R, Li B, Cohen S H, Ching C B, McHugh K M, Becknell B. The uroplakin plaque promotes renal structural integrity during congenital and acquired urinary tract obstruction. Am J Physiol Ren Physiol 2018; 315(4):F1019e31.
[21] Yeh C H, Chiang H S, Lai T Y, Chien C T. Unilateral ureteral obstruction evokes renal tubular apoptosis via the enhanced oxidative stress and endoplasmic reticulum stress in the rat. Neurourol Urodyn 2011; 30(3):472e9.
[22] Cost N G, Noh P H, Devarajan P, Ivancic V, Reddy P P, Minevich E, et al. Urinary NGAL levels correlate with differential renal function in patients with ureteropelvic junction obstruction undergoing pyeloplasty. J Urol 2013; 190(4 Suppl): 1462e7.
[23] Kuwabara T, Mori K, Mukoyama M, Kasahara M, Yokoi H, Saito Y, et al. Urinary neutrophil gelatinase-associated lipocalin levels reflect damage to glomeruli, proximal tubules, and distal nephrons. Kidney Int 2009; 75(3):285e94.
[24] Madsen M G, Norregaard R, Palmfeldt J, Olsen L H, Frokiaer J, Jorgensen T M. Urinary NGAL, cystatin C, beta2-microglobulin, and osteopontin significance in hydronephrotic children. Pediatr Nephrol 2012; 27(11):2099e106.
[25] Nielsen K L, Dynesen P, Larsen P, Jakobsen L, Andersen P S, Frimodt-Moller N. Role of urinary cathelicidin LL-37 and human beta-defensin 1 in uncomplicated Escherichia coli urinary tract infections. Infect Immun 2014; 82(4):1572e8.
[26] Karakus S, Oktar T, Kucukgergin C, Kalelioglu I, Seckin S, Atar A, et al. Urinary IP-10, MCP-1, NGAL, cystatin-C, and KIM1 levels in prenatally diagnosed unilateral hydronephrosis: the search for an ideal biomarker. Urology 2016; 87:185e92.
[27] Miranda E P, Duarte R J, de Bessa Jr J, Lopes R I, Srougi V, Andrade H S, et al. The role of urinary KIM-1, NGAL, CA19-9 and beta2-microglobulin in the assessment of ureteropelvic junction obstruction in adults. Biomarkers 2017; 22(7):682e8.
[28] Wasilewska A, Taranta-Janusz K, Debek W, Zoch-Zwierz W, Kuroczycka-Saniutycz E. KIM-1 and NGAL: new markers of obstructive nephropathy. Pediatr Nephrol 2011; 26(4):579e86.
[29] Xie Y, Xue W, Shao X, Che X, Xu W, Ni Z, et al. Analysis of a urinary biomarker panel for obstructive nephropathy and clinical outcomes. PloS One 2014; 9(11):e112865.
[30] Noyan A, Parmaksiz G, Dursun H, Ezer S S, Anarat R, Cengiz N. Urinary NGAL, KIM-1 and L-FABP concentrations in antenatal hydronephrosis. J Pediatr Urol 2015; 11(5):249 e1e6.

1. A method of detecting a uroepithelial peptide in a patient, wherein the presence of the uroepithelial peptide is indicative of urinary tract obstruction, the method comprising:
(a) obtaining a urine sample from a human patient; and
(b) detecting whether a predetermined uroepithelial peptide is present in the urine sample by contacting the urine sample with an anti-uroepithelial peptide antibody and detecting binding between the uroepithelial peptide and the antibody.

2. The method of claim 1, wherein the uroepithelial peptide is β defensin 1 (BD-1).

3. The method of claim 1, wherein the uroepithelial peptide is human α defensin 5 (HD-5).

4. The method of claim 1, wherein the uroepithelial peptide is hepatocarcinoma-intestine-pancreas/pancreatitis-associated protein (HIP/PAP).

5. The method of claim 1, wherein the uroepithelial peptide is cathelicidin LL-37.

6. The method of claim 1, wherein the uroepithelial peptide is neutrophil gelatinase-associated lipocalin (NGAL).

7. The method of claim 1, further comprising measuring the amount of uroepithelial peptide in the urine sample using quantitative enzyme-linked immunosorbent assay (ELISA).

8. A method of detecting a urinary protein in a patient, wherein the presence of the urinary protein is indicative of urinary tract obstruction, the method comprising:
(a) obtaining a urine sample from a human patient; and
(b) detecting whether a predetermined urinary protein is present in the urine sample by contacting the urine sample with an anti-urinary protein antibody and detecting binding between the urinary protein and the antibody.

9. The method of claim 8, wherein the urinary protein is UPK3A.

10. The method of claim 8, wherein the urinary protein is UPK2.

11. The method of claim 8, wherein the urinary protein is UPK20.

12. The method of claim 8, wherein the urinary protein is UPK14.

13. The method of claim 8, further comprising measuring the amount of urinary protein in the urine sample using quantitative ELISA.

14. A method of diagnosing and treating urinary tract obstruction, comprising:
(a) obtaining a urine sample from a human patient;
(b) detecting whether a predetermined uroepithelial peptide or predetermined urinary protein is present in the urine sample by either contacting the urine sample with an anti-uroepithelial peptide antibody and detecting binding between the uroepithelial peptide and the antibody or contacting the urine sample with an anti-urinary protein antibody and detecting binding between the urinary protein and the antibody;
(c) diagnosing the patient with urinary tract obstruction when the presence of either the predetermined uroepithelial peptide or predetermined urinary protein is detected; and
(d) operatively or non-operatively treating the patient to correct or remove the urinary tract obstruction.

15. The method of claim 14, wherein the uroepithelial peptide is β defensin 1 (BD-1).

16. The method of claim 14, wherein the uroepithelial peptide is human α defensin 5 (HD-5).

17. The method of claim 14, wherein the uroepithelial peptide is hepatocarcinoma-intestine-pancreas/pancreatitis-associated protein (HIP/PAP).

18. The method of claim 14, wherein the uroepithelial peptide is cathelicidin LL-37.

19. The method of claim 14, wherein the uroepithelial peptide is neutrophil gelatinase-associated lipocalin (NGAL).

20. The method of claim 14, wherein urinary protein is UPK3A, UPK2, UPK20, or UPK14.

21. The method of claim 14, further comprising measuring the amount of uroepithelial peptide or the amount of urinary protein in the urine sample using quantitative ELISA.

22. The method of claim 14, further comprising confirming the effective removal of the urinary tract obstruction by obtaining a second, post-treatment urine sample from the patient and testing the second urine sample to confirm an absence of or significant reduction in the amount of urinary protein.
